(19) United States
(12) Patent Application Publication
(10) Pub. No.: US 2003/0082125 A1
(43) Pub. Date: May 1, 2003

(54) Matt cosmetic composition comprising a non-volatile hydrocarbon oil and an inert particulate phase

(76) Inventor: Sabine Grimm, Chatenay Malabry (FR)

Correspondence Address: FINNEGAN, HENDERSON, FARABOW, GARRETT &, DUNNER LLP, 1300 I STREET, NW, WASHINGTON, DC, 20006, US

(21) Appl. No.: 10/111,818

(22) PCT Filed: Aug. 28, 2001
(86) PCT No.: PCT/FR01/02680

(30) Foreign Application Priority Data
Aug. 30, 2000 (FR) 00/11079

(51) Int. Cl. A61K 7/06

(52) U.S. Cl. 424/70.1

(57) ABSTRACT

The present invention relates to a cosmetic care and/or make-up composition for the skin and/or the superficial body growths and/or the lips, which may be provided in the form of an emulsion or an anhydrous gel, comprising a nonvolatile hydrocarbon-based oil having a molecular mass ranging from 230 to 280 g/Mol, a chemically inert particulate phase and optionally a nonvolatile silicone-based compound, compatible with the nonvolatile hydrocarbon-based oil.

The invention also relates to a method for the care of or applying make-up to the skin, the superficial body growths and the lips using such a composition.

More especially, the invention is a fluid make-up product for the skin, having properties of mattness, non-migration and good retention, while being comfortable to wear over time.

The present invention relates to a composition containing a nonvolatile hydrocarbon-based oil and an inert particulate phase containing at least one porous inert filler, intended in particular for the cosmetic field. More especially, the invention relates to a matt care and/or make-up composition for the skin both of the face and of the human body, the lips, the upper or lower eyelids or the superficial body growths such as the eyelashes, the eyebrows and the hair.

This composition may be provided in particular in the form of a cast product as a stick or as a dish, such as lipsticks or lip balms, cast foundations, concealer products, eyeshadows or blushers, in the form of a more or less fluid paste or cream such as fluid foundations or lipsticks, eyeliners, mascaras, colouring compositions or artificial tanning compositions for the skin or make-up compositions for the body or the hair.

Make-up products for the skin or the lips of human beings, such as foundations or lipsticks, generally contain fatty phases such as waxes and oils, pigments and/or fillers and optionally additives such as cosmetic or dermatological active agents. They may also contain so-called “pasty” products, of soft consistency, which make it possible to obtain coloured or noncoloured pastes to be applied with a brush.

When these compositions are applied to the skin or the lips of the face, they have the disadvantage of migrating, that is to say of spreading inside the wrinkles and fine lines of the skin around the lips and the eyes, causing an unaesthetic effect. In addition, they exhibit poor retention over time, and in particular of colour. This poor retention is characterized by an alteration in colour (change in colour, turning pale) generally following an interaction with the sebum and/or the sweat from the skin and, for the lips, an interaction with saliva. Indeed, a composition which is not retained over time forces the user to apply make-up very often. However, up until now, users wished to make their face and their body beautiful while spending the shortest time possible on it.

To increase the retention of a lipstick or of a foundation, the company Shiseido envisaged, in its patent application JP-A-61-65809, make-up compositions containing a siloxysilicate resin (having a three-dimensional network), a volatile silicone oil containing a cyclic silicone chain and pulverulent fillers. These compositions, although having improved retention properties, have the disadvantage of leaving on the skin or the lips, after evaporation of the silicone oils, a film which very rapidly becomes uncomfortable over time (sensation of dryness, of tightness and of discomfort), putting a number of women off this type of make-up product.

However, consumers are still looking for a product, which is comfortable to wear throughout the day, which does not migrate or which scarcely migrates into the skin folds around the lips or the eyes and which exhibits good retention over time.

Furthermore, a matt effect is very often desired for users with combination or greasy skins, and in hot and humid climates. One of the objectives of foundations is to give the skin a good appearance, without conferring on it a greasy, gleaming and shiny appearance. To do this, fillers generally termed mattifying fillers are used, which are most often absorbent fillers such as talc, silica, kaolin or fillers having light scattering optical properties, which properties are known under the name “soft focus” effect. A foundation with mattifying properties is in particular described in the document U.S. Pat. No. 5,288,481 by the company Procter and Gamble.

The higher the quantity of fillers, the greater the matt effect. Unfortunately, the fillers confer on the composition a rough feel and offer a sensation of a “brake” during application which can cause inhomogeneous make-up application, which is not desirable, in particular when the surface to which make-up is to be applied is large; this is the case for the human face or body.

The addition to these filler-rich compositions of a large quantity of volatile silicone, in general greater than 20% of the total weight of the composition, facilitates their application to the skin or the lips and enhances the sensation of slipperiness. However, the sensations of tightness and discomfort then appear.

The need therefore still exists for a composition not exhibiting the above disadvantages and having in particular good properties of slipperiness, retention and nonmigration, while conferring on the deposit a matt appearance which is suitable for the wishes of the consumer, which does not dry out and does not cause tightening of the skin or the lips to which it is applied, both during application and over time.

The applicant has observed, quite surprisingly, that the use of a nonvolatile hydrocarbon-based oil of low molecular mass combined with so-called mattifying fillers, in a physiologically acceptable and more especially cosmetic composition, made it possible to obtain a matt deposit, having good retention, which does not migrate, while being very pleasant during application and to wear throughout the day. The deposit is soft and unctuous. Furthermore, the composition obtained is easy to apply, slippery and without the sensation of a “brake”. Above all, the matt effect exhibits very good retention, that is to say that the mattness is preserved throughout the use and for several hours.

The subject of the present invention is therefore a care or make-up composition for keratin materials, comprising from 5 to 99%, as an amount per mass, of a nonvolatile hydrocarbon-based oil having a molecular mass ranging from 230 to 280 g/Mol and from 3 to 30% of the total weight of the composition of an inert particulate phase containing at least one porous inert filler.

Advantageously, the composition of the invention contains less than 15% by weight of a volatile silicone and, more generally, of a volatile solvent, relative to the total weight of the composition and, even better, less than 10% by weight.

The expression “oil” is understood to mean any nonaqueous medium which is liquid at room temperature (25° C.) and atmospheric pressure (760 mmHg).

The expression “nonvolatile” is understood to mean a medium capable of remaining on the skin or the lips for at least a few hours. A nonvolatile medium has in particular a non-zero vapour pressure, at room temperature and atmospheric pressure, of less than 0.02 mmHg (2.66 Pa).

The expression “volatile” is understood to mean a medium capable of evaporating from the skin or the lips in less than one hour. A volatile medium is in particular chosen from media having a vapour pressure, at room temperature and atmospheric pressure, ranging from 0.02 mmHg to 300 mmHg (2.66 Pa to 40 000 Pa), and more especially from 0.1 to 90 mmHg (13 Pa to 12 000 Pa). In addition, the volatile silicones generally have a viscosity of less than 5 cSt, at room temperature and atmospheric pressure.

The expression inert “particulate phase” is understood to mean any filler which is solid at room temperature and atmospheric pressure, used alone or in combination, which does not react chemically with the various ingredients of the composition and which are insoluble in these ingredients, even when these ingredients are heated to a temperature greater than room temperature.

Thus, the composition may contain one or more nonvolatile hydrocarbon-based oils of low molecular mass.

This composition is in particular a cosmetic or dermatological composition. It therefore contains ingredients which are compatible with keratin materials such as the skin, the lips, the keratin fibres and the nails. It may be provided in the form of an anhydrous gel, an emulsion or an oil-in-water (O/W) or water-in-oil (W/O) dispersion or in the form of a multiple emulsion. It may be provided, in addition, in a more or less fluid form, in the form of a paste or a nondeformable or rigid solid, optionally cast as a stick or as a dish. Preferably, it is provided in fluid form, in particular in the form of an emulsion. The expression “fluid” is understood to mean a composition which flows under its own weight, as opposed to a solid.

The nonvolatile hydrocarbon-based oil(s) of low molecular mass, according to the invention, play the role of nonvolatile solvent. They are provided in particular in the form of an ester resulting in particular from the esterification of acid with C₂ to C₁₅ alcohols, C₂ to C₈ polyols and mixtures thereof. Preferably, the acid is a branched-chain acid such as neopentanoic acid. Thus, the nonvolatile hydrocarbon-based oil comprises in particular a saturated and branched hydrocarbon-based chain.

Advantageously, the nonvolatile hydrocarbon-based oil is chosen from isodecyl neopentanoate (242.4 g/Mol), isotridecyl neopentanoate (270 g/Mol) and mixtures thereof. It is possible to use, however, octyl isononanoate (270.44 g/Mol), isopropyl myristate (270.46 g/Mol), 2-ethylhexyl 2-ethylhexanoate (256.43 g/Mol) and mixtures thereof.

Preferably, the composition comprises, in addition, at least one nonvolatile silicone-based compound. Accordingly, the composition according to the invention therefore advantageously comprises one or more physiologically acceptable nonvolatile silicone-based compounds.

According to the invention, the nonvolatile silicone-based compound and the nonvolatile hydrocarbon-based oil are compatible with each other. Thus, the nonvolatile oil solubilizes or disperses the nonvolatile silicone-based compound. The deposit obtained on the skin is homogeneous, matt, comfortable and soft. It does not migrate into the fine lines around the lips and the eyes in particular.

Advantageously, the composition contains at least one ingredient chosen from cosmetic and dermatological active agents and mixtures thereof. By virtue of the particulate phase and of the particular nonvolatile hydrocarbon-based oil, it is possible to limit, or even eliminate the migration of the compound and in particular of its active agents and/or its colouring matter and therefore to increase retention, over time, of the said composition, while remaining comfortable and matt. The low molecular mass of the hydrocarbon-based oil facilitates its penetration into the skin and the lips.

Another subject of the invention is the use, in a cosmetic composition or for the manufacture of a composition for topical application, of the combination of at least one nonvolatile hydrocarbon-based oil having a molecular mass ranging from 230 to 280 g/Mol and of an inert particulate phase containing at least one porous inert filler, to preserve the mattness of a composition film deposited on the skin and/or the lips of a human being.

The subject of the invention is also a cosmetic method for the care of or the application of make-up to the skin, the lips or the superficial body growths, consisting in applying to the skin, the lips or the superficial body growths, respectively, a cosmetic composition as defined above.

The subject of the invention is also a method for preserving the mattness of a make-up or care composition for the skin or the lips, containing at least one ingredient chosen from colouring matter, cosmetic or dermatological active agents and mixtures thereof, consisting in introducing into the said composition the combination containing at least one nonvolatile hydrocarbon-based oil having a molecular mass ranging from 230 to 280 g/Mol and at least one inert particulate phase as defined above.

It has furthermore been observed that the composition according to the invention exhibits particularly advantageous qualities of spreading and adhesion onto the skin and the lips, as well as an unctuous and pleasant feel. This composition has, in addition, the advantage of being easy to remove as make-up, in particular with a conventional make-up removing milk. This is quite remarkable since the prior art compositions considered as exhibiting long retention are very difficult to remove as make-up, which introduces an additional constraint for the user.

The nonvolatile silicone-based compounds of the invention should be soluble or dispersible in the nonvolatile hydrocarbon-based oil(s) of low molecular mass and in particular in the nonvolatile esters. They are preferably chosen from compounds which are liquid at room temperature, and, still more preferably, they have a viscosity in the range extending from 5 to 1 000 000 cSt at 25° C. and even better from 10 to 500 000 cSt, and even better from 10 to 5 000 cSt.

By way of examples of silicone-based compounds, there may be mentioned polydimethylsiloxanes, phenyltrimethicones, polyalkylmethylsiloxanes, silicone resins such as those described in the documents JP-A-62-61911, JP-A-61-65809 and EP-A-602905, fluorinated silicones and mixtures thereof.

In particular, these silicone-based compounds are chosen from nonvolatile polydimethylsiloxanes (PDMS); polydimethylsiloxanes comprising alkyl, alkoxy or phenyl groups, pendent or at the end of a silicone chain, groups having from 2 to 24 carbon atoms; phenyl trimethicones, phenyl dimethicones, phenyl trimethylsiloxydiphenylsiloxanes, diphenyl dimethicones, diphenyl methyldiphenyltrisiloxanes, 2-phenylethyl trimethylsiloxysilicates; fluorinated silicones comprising a fluorinated group pendent or at the end of a silicone chain having from 1 to 12 carbon atoms in which all or some of the hydrogens are substituted by fluorine atoms; silicone resins; silicone gums; dimethiconols; and mixtures thereof.

The amount by mass of nonvolatile silicone-based compound in the final composition is, for example, in the range extending from 0.5 to 90%, and preferably from 5 to 60%, and still more preferably from 10 to 50%.

The quantities of nonvolatile hydrocarbon-based oils of low molecular mass and of particulate phase are such that they confer on the composition properties of retention, nonmigration, comfort, slipperiness and mattness.

In practice, the nonvolatile hydrocarbon-based oil(s) according to the invention represent in particular an amount by mass from 5 to 99%, preferably from 10 to 70%, and better still from 20 to 50%.

The composition may, in addition, contain at least one additional fatty substance different from the nonvolatile silicone-based compound and the nonvolatile hydrocarbon-based oil, chosen from waxes, gums, fatty substances which are pasty at room temperature, oils and mixtures thereof, this or these fatty substance(s) being of mineral, animal, plant or synthetic origin.

The additional oily fatty substances of the composition may be a cosmetically or dermatologically acceptable, and in general physiologically acceptable, oil chosen in particular from volatile or nonvolatile oils of mineral, animal, plant or synthetic origin.

As additional oils which can be used in the composition according to the invention, there may be mentioned in particular:

hydrocarbon-based oils of animal origin, such as perhydrosqualene;

hydrocarbon-based vegetable oils such as liquid triglycerides of fatty acids of 4 to 24 carbon atoms such as the heptanoic or octanoic acid triglycerides or alternatively sunflower, maize, soybean, gourd, grapeseed, sesame, rapeseed, hazelnut, apricot, macadamia, castor and avocado oils, caprylic/capric acid triglycerides such as those sold by the company Stearineries Dubois or those sold under the names Miglyol 810, 812 and 818 by the company Dynamit Nobel, jojoba oil, shea butter;

linear or branched hydrocarbons of mineral or synthetic origin, such as paraffin oils and derivatives thereof, petroleum jelly, polydecenes, hydrogenated polyisobutene such as Parleam;

synthetic esters and ethers, in particular of fatty acids, such as the oils of formula R₃COOR₄ in which R₃ represents the residue of a higher fatty acid comprising from 1 to 40 carbon atoms and R₄ represents a hydrocarbon chain containing from 1 to 40 carbon atoms with R₃+R₄≧17, such as for example Purcellin oil, isononyl isononanoate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, triisocetyl citrate, heptanoates, octanoates, decanoates of fatty alcohols; polyol esters such as propylene glycol dioctanoate, neopentyl glycol diheptanoate, diethylene glycol diisononanoate; and pentaerythritol esters such as pentaerythrytyl tetraisostearate;

fatty alcohols having from 12 to 26 carbon atoms, such as octyldodecanol, 2-butyloctanol, 2-hexyldecanol, 2-undecylpentadecanol, oleyl alcohol;

partially hydrocarbon-based and/or silicone-based optionally volatile fluorinated oils such as methoxy-nonafluorobutane;

volatile silicone-based oils such as linear or cyclic volatile polydimethylsiloxanes (PDMS) having from 2 to 7 silicon atoms, these silicones optionally comprising alkyl or alkoxy groups having from 1 to 10 carbon atoms pendent or at the end of a silicone-based chain such as octamethylcyclotetrasiloxane, decamethylcyclopentasiloxane, hexadecamethylcyclohexasiloxane, heptamethylhexyltrisiloxane, heptamethyloctyltrisiloxane and mixtures thereof;

mixtures thereof.

The additional nonvolatile oil(s) of the composition may represent from 0.1% to 90% of the total weight of the composition, and preferably from 5 to 60%.

The composition contains, in addition, an inert particulate phase containing at least one inert filler, absorbent or otherwise, that is to say one or more inert fillers, in particular absorbing oils and/or mattifying. Preferably, these fillers have an apparent diameter ranging from 0.01 to 150 μm, and better still from 0.5 to 150 μm. An apparent diameter corresponds to the diameter of the circle in which the elementary particle is inscribed along its smallest dimension (thickness for leaflets).

The fillers may be inorganic or organic, lamellar, spherical or oblong. The fillers are generally porous in order to absorb the sweat and/or the sebum secreted by the skin, such as for example silica, polyethylene powder, Nylon®, kaolin, starch derivatives, porous acrylic copolymers such as Polytrap® and porous polyalkyl methacrylates such as Polypore E 200® or Polypore L 200®.

The particulate filler may advantageously represent from 3 to 30% of the total weight of the composition, preferably from 5 to 25%, and even better from 10 to 20%.

The composition of the invention generally comprises at least one colouring matter. The said colouring matter advantageously contains at least (one or more) pulverulent colouring compounds and/or one or more fat-soluble or water-soluble colorants, for example in an amount of 0.01 to 70% of the total weight of the composition and in particular from 0.05 to 70%. The pulverulent colouring compound(s) may be chosen from the pigments and pearlescent agents normally used in cosmetic or dermatological compositions, and mixtures thereof. Advantageously, the pulverulent colouring compounds represent up to 50% of the total weight of the composition, in particular from 0.1 to 50%, and even better from 1 to 40%.

The pigments may be white or coloured, inorganic and/or organic, interferential or otherwise, insoluble in the liquid fatty phase, intended to colour and/or opacify the composition. There may be mentioned, among the inorganic pigments, titanium dioxide, optionally surface-treated, zirconium or cerium oxides, as well as zinc, iron or chromium oxides, manganese violet, ultramarine blue, chromium hydrate and ferric blue. Among the organic pigments, there may be mentioned carbon black, pigments of the D & C type, and lacquers based on carmine, barium, strontium, calcium, aluminium.

The pearlescent pigments may be chosen from white pearlescent pigments such as mica coated with titanium or bismuth oxychloride, coloured pearlescent pigments such as mica-titanium with iron oxides, mica-titanium with in particular ferric blue or chromium oxide, mica-titanium with an organic pigment of the abovementioned type as well as pearlescent pigments based on bismuth oxychloride.

The fat-soluble colorants are, for example, Sudan red, DC Red 17, DC Green 6, β-carotene, soybean oil, Sudan brown, DC Yellow 11, DC Violet 2, DC orange 5, quinoline yellow. They may represent from 0 to 20%, and in particular 0.01 to 20% of the weight of the composition, and even better from 0.1 to 6%. The water-soluble colorants are, for example, beet juice, methylene blue and may represent up to 6% of the total weight of the composition.

The composition of the invention may, in addition, contain one or more cosmetic or dermatological active agents such as those conventionally used.

As cosmetic or dermatological active agents which can be used in the composition of the invention, there may be mentioned moisturizing agents such as polyols, vitamins, essential fatty acids, sphingolipids, sunscreens. These active agents are used in a quantity which is customary for persons skilled in the art and in particular at concentrations from 0 to 20%, and in particular from 0.001 to 20% of the total weight of the composition.

The composition according to the invention may, furthermore, comprise, according to the type of application envisaged, the constituents conventionally used in the fields considered, which are present in a quantity appropriate for the desired galenic form.

Among the waxes solid at room temperature, which may be present in the composition according to the invention, there may be mentioned hydrocarbon-based waxes such as optionally modified beeswax, Carnauba, Candellila, Ouricoury or Japan wax, cork fibre or sugar cane waxes, paraffin or lignite waxes, microcrystalline waxes, lanolin wax, Montan wax, ozokerites, polyethylene waxes, the waxes obtained by Fischer-Tropsch synthesis and C₂₀-C₆₀ fatty alcohols. It is also possible to use silicone waxes, among which there may be mentioned alkyl- and/or alkoxypolymethylsiloxanes and/or polymethylsiloxane esters and mixtures thereof.

The waxes may be present in an amount of 0-50% (for example from 0.01 to 50%) by weight in the composition, and even better from 5 to 20%.

As pasty fatty substances, there may be mentioned fatty substances having a melting point ranging from 25 to 45° C. and/or a viscosity at 40° C. ranging from 0.1 to 40 Pa.s measured using a Contraves TV device equipped with an MS-r3 or MS-r4 rotor rotating at 60 Hz. By way of example of pasty fatty substances, there may be mentioned PDMSs having pendent chains of the alkyl or alkoxy type having from 8 to 24 carbon atoms such as stearyl dimethicone; esters of fatty alcohol or of fatty acid such as cholesterol esters, polyvinyl laurate, arachidyl propionate; PVP/Eicosene copolymers; lanolins and derivatives thereof such as acetylated lanolins or oxypropylenated lanolins; and mixtures thereof.

The nature and the quantity of the waxes, pasty fatty substances and gums depend on the mechanical properties and textures desired.

In a particular embodiment of the invention, the compositions according to the invention may be prepared in the customary manner by persons skilled in the art. They may be provided in the form of a cast product and, for example, in the form of a stick or in the form of a dish which can be used by direct contact or with a sponge or alternatively in a boiling pan. In particular, they find application as cast foundation, cast blusher or eyeshadow, lipstick, care base or balm for the lips, and concealer product. They may also be provided in the form of a soft paste or alternatively a gel or a more or less fluid cream. They can also constitute foundations or lip gloss, antisun products or skin colouring products.

The compositions of the invention advantageously contain an aqueous phase. They may then be provided in particular in the form of a simple or multiple emulsion having an oily or aqueous continuous phase, an oily dispersion in an aqueous phase by virtue of vesicles containing ionic and/or nonionic lipids or in the form of a vesicular dispersion containing ionic and/or nonionic lipids. They may also be provided in the form of an oily gel, an oily liquid, a paste or a stick. These galenic forms are prepared according to the customary methods in the fields considered.

Preferably, the composition according to the invention constitutes a make-up composition, in particular for the skin and more especially a foundation. This foundation is perfectly suitable for greasy or combination skins and for hot and humid climates.

Preferably, the composition is provided in the form of an emulsion, that is to say a composition containing an aqueous phase and a liquid fatty phase in which one of the two phases is dispersed in the other phase with or without emulsifier, the whole being homogeneous to the naked eye.

The aqueous phase is a phase which is immiscible with the hydrocarbon-based and silicone-based oils mentioned above, containing water optionally thickened or gelled with one or more aqueous phase thickening agents or gelling agents and optionally containing water-miscible compounds, such as C₂ to C₇ lower alcohols, polyols having at least two hydroxyl groups and 2 to 10 carbon atoms such as glycerol, propylene glycol, D-panthenol, polyethylene glycols. The aqueous phase represents in particular 0.1 to 90% of the total weight of the composition, preferably 5 to 80%, and even better from 15 to 70%.

The emulsion according to the invention may be obtained using an emulsifying agent or a mixture of emulsifying agents whose HLB (hydrophilic/lipophilic balance) is adjusted in the direction of the emulsion.

As emulsifier which can be used in the invention, suitable for obtaining a W/O emulsion, there may be mentioned those having an HLB of less than 7 and in particular fatty acid esters of polyols such as mono-, di-, tri- or sesqui-oleates or -stearates of sorbitol or glycerol, laurates of glycerol or polethylene glycol; alkyl or alkoxy dimethicone copolyols having an alkyl or alkoxy chain pendent or at the end of a silicone-based backbone having for example from 6 to 22 carbon atoms. As emulsifiers which can be used in the invention for producing an O/W emulsion, there may be mentioned those having an HLB greater than 7 such as fatty acid esters of polyethylene glycol (monostearate or monolaurate of polyethylene glycol); esters of fatty acids (stearate, oleate) of sorbitol which are polyoxyethylenated; polyoxy ethylenated alkyl (lauryl, cetyl, stearyl, octyl) ethers and dimethicone copolyols. In general, it is possible to use any amphoteric ionic (cationic or anionic) emulsifier or any nonionic emulsifier well known to persons skilled in the art.

The composition of the invention may comprise, in addition, any additive customarily used in the field considered, chosen in particular from surfactants, oil thickeners, antioxidants, essential oils, preservatives, perfumes, electrolytes (cation or polycations), fillers, waxes, products which are pasty at room temperature, neutralizing agents, polymers which are fat-soluble or dispersible in the medium, cosmetic or dermatological active agents such as, for example, emollients, moisturizers, vitamins, essential fatty acids, sunscreens, dispersants such as poly(12-hydroxystearic) acid and mixtures thereof. These additives may be present in the composition in an amount of 0 to 20% (in particular from 0.01 to 20%) of the total weight of the composition and even better from 0.01 to 10%. Advantageously, the composition contains at least one cosmetic or dermatological active agent. Of course, persons skilled in the art will be careful to choose this or these optional additional compounds and/or their quantity so that the advantageous properties of the composition according to the invention are not, or not substantially, impaired by the addition envisaged.

These compositions for topical application may constitute in particular a cosmetic or dermatological protective, treatment or care composition for the face, for the neck, for the hands or for the body (for example a care cream, antisun oil, body gel), a make-up composition (for example make-up gel, cream, stick) or an artificial tanning composition or skin-protecting composition.

The invention is illustrated in more detail in the following examples. The percentages are percentages by weight.

### EXAMPLE 1

### O/W Type Fluid Foundation

| | |
|---|---|
| Isodecyl neopentanoate | 13.50% |
| Coated yellow iron oxide | 1.4% |
| Coated brown iron oxide | 0.5% |
| Coated black iron oxide | 0.2% |
| Coated titanium oxide anatase | 3.9% |
| Butyl p-hydroxybenzoate | 0.15% |
| Methyl p-hydroxybenzoate | 0.4% |
| Magnesium sulphate | 1.0% |
| Nylon 12 powder | 6.0% |
| Polyethylene powder | 4.0% |
| Mixture of crosslinked polyoxyethylenated | 12.5% |
| PDMS and of PDMS of 6 cSt | |
| Phenyl trimethylsiloxytrisiloxane (20 cSt) | 9.0% |
| Mixture of PDMS having alpha-omega hydroxyl | 3.0% |
| groups and of PDMS 5 cSt | |
| Polymethylcetyldimethylsiloxane | 6.0% |
| (MW*: 900-viscosity: 15-25 cSt) | |
| Propylene glycol | 3.0% |
| Hexaglycerol | 2.0% |
| Polyethylene glycol (20 EO)** | 1.7% |
| Polyethylene glycol (8 EO)** | 3.0% |
| Demineralized water | qs 100% |

*MW means number-average molecular mass
**EQ means ethylene oxide

### EXAMPLE 2

### O/W Type Fluid Foundation

| | |
|---|---|
| Isodecyl neopentanoate | 14.0% |
| Polyethylene glycol (32 EO)** | 9.3% |
| Polyethylene powder | 9.3% |
| Propylene glycol | 6.5% |
| Titanium dioxide | 5.6% |
| Stearic acid | 1.9% |
| Mixture of yellow, brown and black iron | 1.4% |
| oxides | |
| Magnesium and aluminium silicate | 1.1% |
| Triethanolamine | 0.9% |
| Polyethylene glycol stearate | 0.6% |
| Glycerol stearate | 0.3% |
| Sodium lauroyl sarcosinate | 0.2% |
| Methyl p-hydroxybenzoate | 0.1% |
| Butyl p-hydroxybenzoate | 0.1% |
| Ethyl p-hydroxybenzoate | 0.6% |
| Glycerine | 0.02% |
| Propyl p-hydroxybenzoate | 0.02% |
| Water | qs 100% |

**EO means ethylene oxide

The foundations of Examples 1 and 2 give a make-up which is mattifying, comfortable, easy to apply, slippery and nonfatty. The colour of the make-up is retained well over time. Migration into the wrinkles around the eyes is low.

### EXAMPLE 3

### O/W Fluid Foundation

The isodecyl neopentanoate of the foundation of Example 2 is replaced with isodridecyl neopentanoate. The properties of mattness, comfort, retention and application of these two foundations are identical.

### Counterexample 1

### O/W Foundation

The foundation of Example 2 was compared with a foundation according to the prior art containing, in place of isodecyl neopentanoate, a cyclopentadimethylsiloxane (6 cSt), by a panel of experts. The two foundations were judged as conferring an identical matt appearance on the skin of the face, with a sensation of slipperiness and of comfort twice as high for the foundation of the invention compared with that of the prior art and an increased retention of the colour over time.

### Counterexample 2

### O/W Foundation

The foundation of Example 2 was compared with a foundation in which the isodecyl neopentanoate was replaced with isononyl isononanoate. The properties of retention and mattness were judged to be better by a panel of experts for the foundation of Example 2 compared with that of Counterexample 2.

1. Care or make-up composition for keratin materials, comprising at least one nonvolatile hydrocarbon-based oil having a molecular mass ranging from 230 to 280 g/Mol, at least one colouring matter and an inert particulate phase.

2. Composition according to claim 1, characterized in that the nonvolatile hydrocarbon-based oil is an ester.

3. Composition according to claim 1 or 2, characterized in that the nonvolatile hydrocarbon-based oil comprises a saturated and branched hydrocarbon-based chain.

4. Composition according to one of the preceding claims, characterized in that the nonvolatile hydrocarbon-based oil is a branched neopentanoic acid ester.

5. Composition according to one of the preceding claims, characterized in that the nonvolatile hydrocarbon-based oil is chosen from C₂ to C₁₅ alcohol esters, C₂ to C₈ polyol esters and mixtures thereof.

6. Composition according to one of the preceding claims, characterized in that the nonvolatile hydrocarbon-based oil is chosen from isodecyl neopentanoate, isotridecyl neopentanoate, octyl isononanoate, isopropyl myristate, 2-ethylhexyl 2-ethyl hexanoate and mixtures thereof.

7. Composition according to one of the preceding claims, characterized in that the nonvolatile hydrocarbon-based oil is chosen from isodecyl neopentanoate, isotridecyl neopentanoate and mixtures thereof.

8. Composition according to one of the preceding claims, characterized in that the nonvolatile hydrocarbon-based oil represents an amount by mass of 5 to 99%, preferably from 10 to 70%, and even better, from 20 to 50%.

9. Composition according to one of the preceding claims, characterized in that the particulate phase contains at least one absorbent and/or mattifying inert filler.

10. Composition according to one of the preceding claims, characterized in that the inert filler is chosen from spherical, lamellar, oblong fillers and mixtures thereof.

11. Composition according to claim 9 or 10, characterized in that the inert filler is chosen from talc, mica, silica, kaolin, powders of polyamide, of poly-β-alanine and of polyethylene, powders of polytetrafluoroethylene, lauroyllysine, starch, boron nitride, polymeric hollow microspheres, acrylic acid copolymers, microbeads of silicone resin, precipitated calcium carbonate, magnesium carbonate and hydrocarbonate, hydroxyapatite, hollow microspheres of silica, microcapsules of glass or of ceramic and mixtures thereof.

12. Composition according to any one of claims 9 to 11, characterized in that the filler has an apparent diameter ranging from 0.01 to 150 μm.

13. Composition according to any one of claims 9 to 12, characterized in that the filler has an apparent diameter ranging from 0.5 to 150 μm.

14. Composition according to one of the preceding claims, characterized in that the particulate phase represents from 3 to 30% of the total weight of the composition, preferably from 5 to 25%, and, even better, from 10 to 20%.

15. Composition according to one of the preceding claims, characterized in that it contains less than 15% by weight of volatile oil, relative to the total weight of the composition and, even better, less than 10% by weight.

16. Composition according to one of the preceding claims, characterized in that it comprises, in addition, at least one nonvolatile silicone-based compound.

17. Composition according to claim 16, characterized in that the nonvolatile silicone-based compound is chosen from compounds which are liquid at room temperature.

18. Composition according to claim 16 or 17, in which the nonvolatile silicone-based compound has a viscosity chosen from the range extending from 5 to 1 000 000 cSt and preferably extending from 10 to 500 000 cSt.

19. Composition according to one of claims 16 to 18, in which the nonvolatile silicone-based compound is chosen from nonvolatile polydimethylsiloxanes (PDMS); polydimethylsiloxanes comprising alkyl, alkoxy or phenyl groups, pendent or at the end of a silicone chain, groups having from 2 to 24 carbon atoms; phenyl trimethicones, phenyl dimethicones, phenyl trimethylsiloxydiphenylsiloxanes, diphenyl dimethicones, diphenyl methyldiphenyltrisiloxanes, 2-phenylethyl trimethylsiloxysilicates; fluorinated silicones comprising a fluorinated group pendent or at the end of a silicone chain having from 1 to 12 carbon atoms in which all or some of the hydrogens are substituted by fluorine atoms; silicone resins; silicone gums; dimethiconols; and mixtures thereof.

20. Composition according to one of claims 16 to 19, characterized in that the nonvolatile silicone-based compound represents an amount by mass from 0.5 to 90%, preferably from 5 to 60%, and, even better, from 10 to 50%.

21. Composition according to one of the preceding claims, in which at least one ingredient chosen from cosmetic active agents, dermatological active agents and mixtures thereof is provided.

22. Composition according to one of claims 6 to 21, comprising, in addition, at least one additional fatty substance different from the nonvolatile silicone-based compound and the nonvolatile hydrocarbon-based oil, chosen from waxes, gums, fatty substances which are pasty at room temperature, oils, emulsifying agents and mixtures thereof.

23. Composition according to one of the preceding claims, in which the colouring matter comprises at least one pulverulent colouring compound chosen from pigments, pearlescent agents and mixtures thereof.

24. Composition according to claim 23, characterized in that the pulverulent colouring compound represents up to 50% of the total weight of the composition.

25. Composition according to one of the preceding claims, provided in the form of a stick, in the form of a soft paste, in the form of a dish, an oily gel, an oily liquid, a vesicular dispersion containing ionic and/or nonionic lipids, a water-in-oil or oil-in-water emulsion or a multiple emulsion.

26. Composition according to one of the preceding claims, characterized in that it contains at least one aqueous phase.

27. Composition according to one of the preceding claims, characterized in that it is provided in the form of an emulsion.

28. Composition according to either of claims 26 and 27, characterized in that the aqueous phase represents from 0.1 to 90% of the total weight of the composition, preferably from 5 to 80%, and, even better, from 15 to 70%.

29. Composition according to one of the preceding claims, provided in the form of a make-up composition.

30. Composition according to one of the preceding claims, provided in the form of a foundation, a blusher or an eyeshadow, a lipstick, a care base or balm for the lips, a concealer product, an eyeliner, a mascara.

31. Skin make-up composition and more particularly a foundation, comprising at least one nonvolatile hydrocarbon-based oil having a molecular mass ranging from 230 to 280 g/Mol, at least one colouring matter and an inert particulate phase.

32. Method of cosmetic care of or make-up application to the lips or the skin, consisting in applying to the lips or the skin, respectively, a cosmetic composition as defined in the preceding claims.

33. Use, in a cosmetic composition or for topical application, of the combination of at least one nonvolatile hydrocarbon-based oil having a molecular mass ranging from 230 to 280 g/Mol and of an inert particulate phase, to increase the retention of a composition film deposited on the skin and/or the lips of a human being and/or to preserve its mattness and/or to impart comfort to the said film, and/or to limit its migration and/or to provide slipperiness and/or to improve the properties of application of the composition.

34. Use according to claim 33, characterized in that the composition contains at least one nonvolatile silicone-based compound.

35. Method for increasing the retention of a make-up or care composition for the skin or the lips and/or increasing the mattness and/or the comfort of the said composition and/or reducing migration of this composition and/or improving its properties of application, containing at least one ingredient chosen from cosmetic or dermatological active agents, colouring matters and mixtures thereof, consisting in introducing into the composition the combination containing at least one nonvolatile hydrocarbon-based oil having a molecular mass ranging from 230 to 280 g/Mol and an inert particulate phase.
